(19) United States
(12) Patent Application Publication
(10) Pub. No.: US 2017/0211876 A1
(43) Pub. Date: Jul. 27, 2017

(54) METHOD FOR SELECTING ANTIBODIES WITH MODIFIED FCRN INTERACTION

(71) Applicant: HOFFMANN-LA ROCHE INC., Little Falls, NJ (US)

(72) Inventors: Hubert KETTENBERGER, Muenchen (DE); Maximiliane KOENIG, Pullach (DE); Tilman SCHLOTHAUER, Penzberg (DE); Pernille FOGED JENSEN, Taastrup (DK); Kasper RAND, Frederiksberg (DK)

(21) Appl. No.: 15/317,324

(22) PCT Filed: Jun. 10, 2015
(86) PCT No.: PCT/EP2015/062899

§ 371 Date: Dec. 8, 2016

(30) Foreign Application Priority Data
Jun. 12, 2014 (EP) 14172180.3

(51) Int. Cl. F25D 31/00

(52) U.S. Cl. CPC F25D 31/002; F25D 2700/16; F25D 2600/04

(57) ABSTRACT

Herein is reported a method for selecting a full length antibody comprising the steps of a) generating from a parent full length antibody a plurality of full length antibodies by randomizing one or more amino acid residues selected from the amino acid residues at positions 1-23 in the heavy chain variable domain (numbering according to Kabat), at positions 55-83 in the light chain variable domain (numbering according to Kabat), at positions 145-174 in the first heavy chain constant domain (numbering according to EU index), and at positions 180-97 in the first heavy chain constant domain (numbering according to EU index), b) determining the binding strength of each of the full length antibodies from the 10 plurality of antibodies to the human neonatal Fc receptor (FcRn), and c) selecting a full length antibody from the plurality of full length antibodies that has a different binding strength to the FcRn than the parent full length antibody.

### CROSS-REFERENCE TO RELATED APPLICATIONS

This application claims priority to U.S. Provisional Application No. 62/090,062, filed Dec. 10, 2014, the content of which is hereby incorporated by reference as if fully recited herein.

### TECHNICAL FIELD

Exemplary embodiments relate to devices and methods for chilling water within a water cooler. A preferred exemplary embodiment comprises a water reservoir wherein at least part of an evaporator coil connected to a compressor driven refrigeration unit is disposed along a helical path within an interior portion of the water reservoir. The helical path is preferably defined at least in part by a helical fin/baffle that is connected to or part of an interior wall of the reservoir. In the preferred embodiment, the helical path is travelled by water as water is drawn from the reservoir, exposing the water to a substantial surface area of the evaporator coil and chilling the water as it travels to the water outlet of the water cooler.

### BACKGROUND AND SUMMARY OF THE INVENTION

Water coolers have become common products found in offices, hospitals, schools and homes. There are two main types of water coolers: bottled water coolers and bottleless water coolers. Both types of water coolers provide chilled water, but they receive water from different sources. Bottled water coolers are freestanding units that use a large plastic bottle to deliver water and come in top-loaded and bottom-loaded varieties. Bottleless water coolers on the other hand hook up to a plumbed water supply and utilize filtration services to provide clean, crisp-tasting water.

Both bottled water coolers and bottleless water coolers have a reservoir that holds a certain amount of water. The reservoir is where the water is chilled prior to being dispensed. In most water coolers, refrigerant (such as Freon for example) is utilized in conjunction with a compressor as a means of chilling the water in the reservoir. In the known cooling tank system, an evaporator coil which maintains the refrigerant is wrapped around the outside of a stainless steel tank. The interior surface of the steel tank defines the water reservoir for the water cooler. Thus, the evaporator coil is separated from the water by the stainless steel tank wall and it is the contact of the evaporator coil with the exterior surface of the tank wall that provides for heat transfer. The warm liquid within the reservoir is cooled as heat passes from the liquid, through the steel reservoir wall, through the walls of the evaporator coil and into the refrigerant within the coil. In this system, heat transfer is occurring at just 3 to 7 percent of the surface area of the reservoir and the evaporator coil respectively. The tank wall, the material it is made from, and the thickness of the wall can impede heat transfer. The intake of water into and the dispensing of water from prior art water reservoirs took place at opposite ends of the reservoir.

The present invention provides a more efficient and effective means for cooling water within a water reservoir of a water cooler and is generally more affordable than prior art systems by, amongst other things, making the evaporator coil an integral part of the water reservoir. A preferred exemplary embodiment of a cooling tank of the present invention comprises an external tank body connected to an external tank cap. An internal tank wall is disposed within the external tank body and is held in a desired location within the external tank body at least in part by a connection to the external tank cap. The internal tank body may comprise a plurality of ribs which engage with part of the external tank cap forming the securing connection. A helical path existing between the internal tank wall and the external tank body houses at least part of an evaporator coil that is connected to a refrigeration system such as a compression driven refrigeration system. The helical path is positioned and adapted to receive water from a water inlet tube that is connected to the tank's water inlet. Under normal operating conditions, water received by the tank will travel and be stored in the helical path being exposed to and chilled by the evaporator coil until the water is removed from the tank via the tank's water outlet.

### BRIEF DESCRIPTION OF THE DRAWINGS

Novel features and advantages of the present invention, in addition to those mentioned above, will become apparent to those skilled in the art from a reading of the following detailed description in conjunction with the accompanying drawings wherein identical characters refer to identical parts and in which:

FIG. 1 is a front perspective view of a first exemplary embodiment of a helical cooling tank;

FIG. 2 is a front section view of the exemplary helical cooling tank shown in FIG. 1;

FIG. 3 is a front section view of a schematic of water flow through the exemplary tank shown in FIGS. 1 and 2 wherein arrows are utilized to indicate the direction of water flowing through the tank when it is in operation.

FIG. 4 is an exploded view of the exemplary helical cooling tank shown in FIG. 1, FIG. 2, and FIG. 3;

FIG. 5 is a front perspective view of an exemplary inner tank wall of a helical cooling tank where said wall has an integrated internal cap and helical fin and wherein said internal tank wall defines a plurality of holes for positioning of a sensor well within the tank;

FIG. 6 is a front section view of a helical cooling tank which incorporates the inner tank wall shown in FIG. 5;

FIG. 7 is an exploded view of a plumbed water cooler shown in conjunction with an exemplary helical cooling tank; and

FIG. 8 is a front perspective view of a second exemplary embodiment of a helical cooling tank;

FIG. 9 is a front section view of the exemplary helical cooling tank shown in FIG. 8;

FIG. 10 is a front section view of a schematic of water flow through the exemplary tank shown in FIGS. 8 and 9 wherein arrows are utilized to indicate the direction of water flowing through the tank when the tank is in operation;

FIG. 11 is an exploded view of the exemplary helical cooling tank shown in FIG. 8, FIG. 9, and FIG. 10;

FIG. 12 is a front perspective view of a preferred exemplary inner tank wall of a helical cooling tank where said wall has an integrated internal cap and helical fin; and

FIG. 13 is a front section view of an exemplary helical cooling tank which incorporates the inner tank wall shown in FIG. 12.

### DETAILED DESCRIPTION OF EXEMPLARY EMBODIMENT(S)

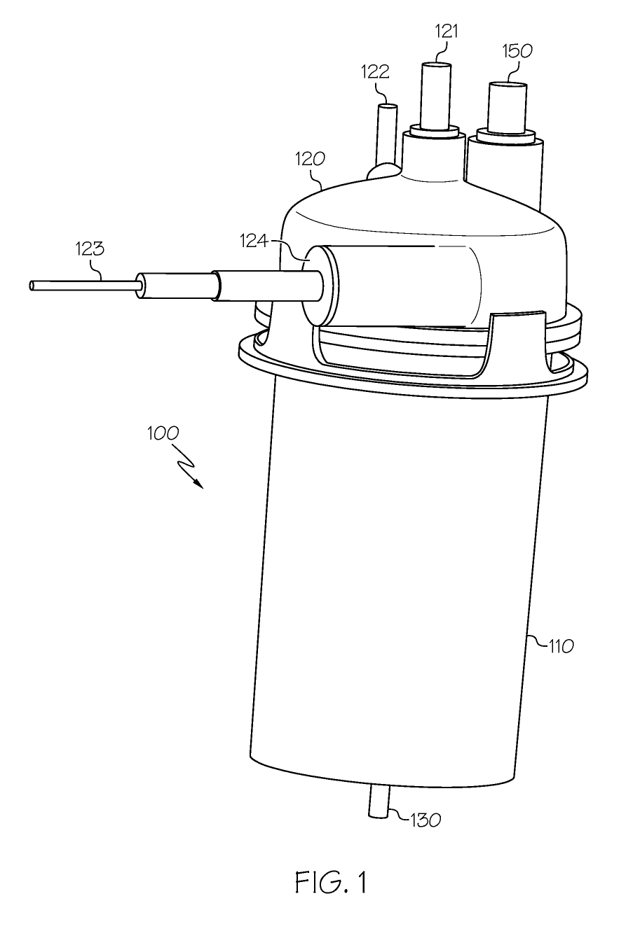

A first and preferred exemplary embodiment of a cooling tank 100 of the present invention is depicted in FIG. 1 wherein external tank body 110 is connected to external cap 120. In some exemplary embodiments, the external tank body 110 and external cap 120 may be a single, integrated unit/tank. In such embodiments, the external tank body 110 may comprise a selectively removable bottom making access to the interior of the tank 100 possible. As shown in FIG. 1, the external cap 120 preferably receives a water inlet 121, a water outlet 122, and a refrigeration inlet/outlet 123. Though not shown in the figure, in operation within a water cooler, water inlet 121 would be connected to and receive water from a water source such as a bottle of water or a plumbed water line and permit for water to be received by the cooling tank 100. Similarly, water outlet 122 would be connected to a water dispensing means (such as a faucet) of the water cooler which would permit for chilled water to be removed from the cooling tank 100 for consumption or other use. Preferably, the refrigeration inlet/outlet 123 is connected to a compressor driven refrigeration system which supplies refrigerant to and receives refrigerant from the cooling tank 100 when water within the cooler is above a desired temperature. The external tank body 110 may define, comprise, and/or be connected to a service drain 130, which permits for water to be drained from the cooling tank when the tank needs to be serviced, the drain 130 is preferably located at the bottom of tank 100. A top cap nut 124 may be utilized to secure the refrigeration inlet/outlet 123 within the external cap 120.

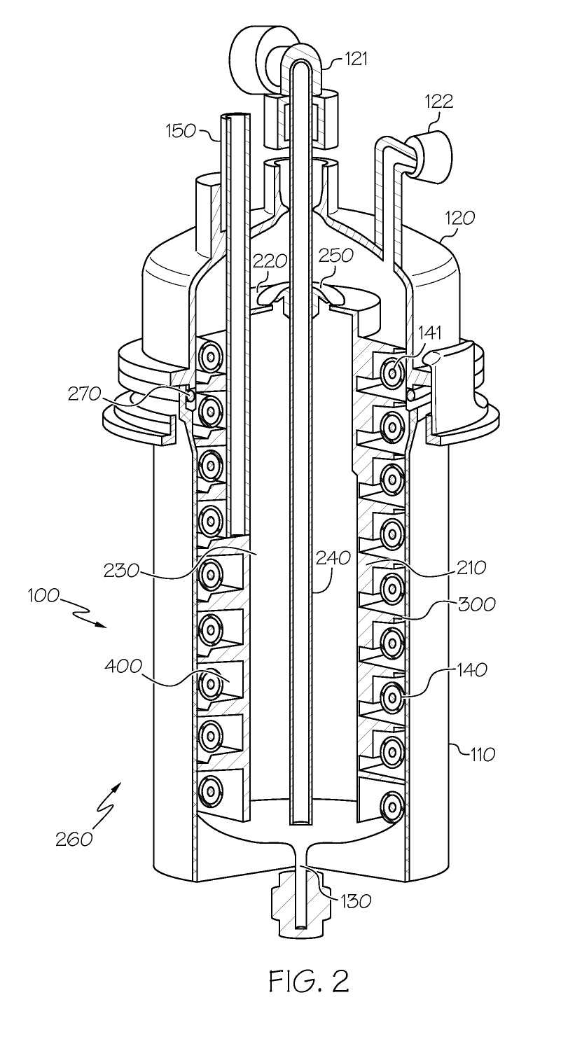

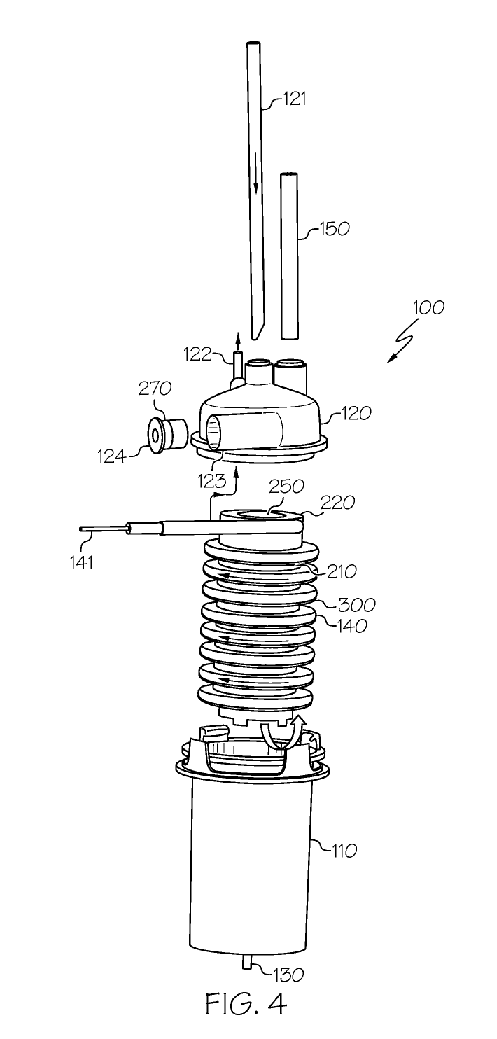

FIG. 2 shows a front section view of the exemplary cooling tank 100 of FIG. 1. As shown, inner tank wall 210 comprising helical fin/baffle 300 is disposed within the external tank body 110. In the preferred embodiment shown in FIG. 2, external cap 120 is connected to both the inner tank wall 210 and the external tank body 110 securing inner tank wall 210 in a desired location within the external tank body 110. It is also preferable that the inner tank wall 210, when positioned within the external tank body 110, comes into contact with the bottom of the external tank body as shown in FIG. 2. An O-ring seal 270, preferably made of a polymeric material, may be positioned between the external cap 120 and the external tank body 110 providing a water tight seal. As shown in FIG. 2, when the inner tank wall 210 comprising helical fin 300 is disposed within the external tank body 110, helical fin 300 preferably seals to the interior surface of the external tank body 110. This is preferably a press fit seal. In such a configuration, the internal tank wall 210, helical fin 300, and external tank body 110 define a helical pathway 400 which receives at least part of an evaporator coil 140 as well as a volume of water. The evaporator coil 140 is wrapped around the internal tank wall 210 within the helical path 400 and receives refrigerant from and returns refrigerant to a refrigeration system that is not shown in the Figure. Also not visible in FIG. 2 is the refrigeration inlet/outlet 123 of exterior cap 120 which permits for refrigerant to be provided to and returned from the evaporator coil 140. Evaporator coil 140 is preferably coaxial, with an inner tube delivering liquid refrigerant which then is expanded and vaporized in an annular space between the respective tubes. In a preferred exemplary Embodiment, as shown in FIG. 2, evaporator coil 140 is wrapped eight and a half turns around the inner tank wall 210. As can be seen in FIG. 4, a refrigeration system capillary tube 141 may be disposed within the evaporator coil 140. In such an embodiment, the liquid refrigerant enters the capillary tube 141 under higher pressure. The pressure is high enough that the refrigerant remains a liquid. As it flows through the capillary tube 141, the restriction caused by the tiny cross section creates a pressure drop. By the time the liquid refrigerant exits the capillary tube 141 into the annular space, the pressure is so low that it expands and begins to boil as it absorbs heat. The temperature of the liquid refrigerant at the capillary exit drops well below the freezing point of water.

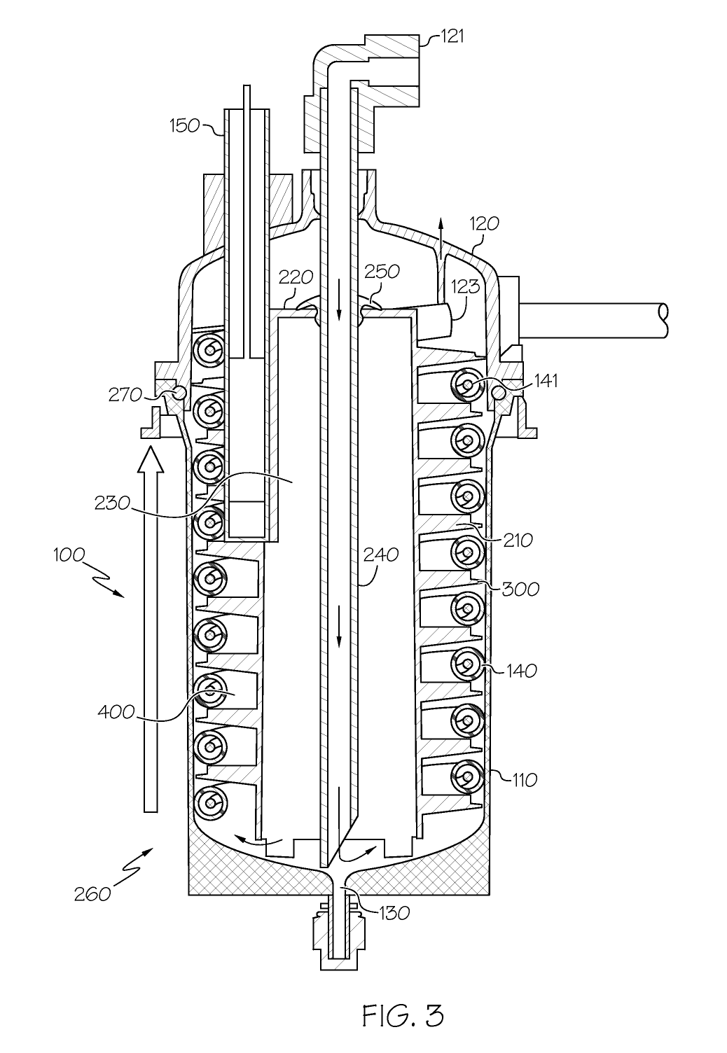

In preferred exemplary embodiments, and as can be seen in FIG. 2, water inlet 121 is connected to a water inlet tube 240 which extends through the external cap 120 as well as the internal tank wall 210 and directs a flow of water to be received by the helical pathway 400 which houses evaporator coil 140. Accordingly, once water is received by the helical pathway 400, it is exposed to and may be chilled by the evaporator coil 140. As water is drawn from tank/reservoir 100 via outlet 122 (for consumption, etc.) the water is, under normal operating conditions, drawn from the volume of water that has been held at least for some period of time within the helical pathway 400. This causes more water to be drawn into the tank 100 via the water inlet 121 and inlet tube 240 and creates a flow of water within the helical pathway 400 along the evaporator coil 140. The schematic provided in FIG. 3 illustrates with arrows how water would flow into, through, and out of the exemplary tank 100 shown in FIGS. 1 and 2. When the compressor driven refrigeration system connected to tank 100 is in operation, the evaporator coil 140 will be cooled via refrigerant and will draw heat from the water within the tank 100. The water becomes colder as it moves along/is stored in the helical path 400 due to exposure to evaporator coil 140. This is also illustrated in the FIG. 3 schematic.

As can be seen in FIG. 2, in a preferred exemplary embodiment, the external tank 110 is in the nature of a can having a generally closed bottom and a generally open top. Similarly, the internal tank 210 is in the nature of a can having a generally open bottom and a generally closed top. The generally closed top of internal tank 210 may comprise an internal cap 220 that is a separate piece connected to wall of the internal tank 210. In other exemplary embodiments, such as those shown in FIGS. 2, 3, and 5, the generally closed top of internal tank 210 may comprise an internal cap 220 that is integral with the wall of internal tank 210. In the preferred exemplary configuration of the cooling tank 100 shown in FIG. 2, the internal tank 210 is inserted into the external tank 110. External cap 120 is connected to both the external tank 110 and the internal tank 210 and assists with holding the internal tank 210 in the desired position within the external tank. Preferably, and as is shown in FIG. 2, a helical fin 300 is supported in place between the external tank 110 and the internal tank 210. As shown in FIG. 2, the fin is supported in place by being part of the internal tank 210, but the fin 300 could be supported in place by a variety of means including by being part of the external tank 110, by being a separate piece that has a press-fit seal between the internal tank 210 and external tank 110, etc.

As shown in FIG. 2, Inner tank wall 210 preferably defines a temporary storage space 230 which is adapted to hold a volume of water in the event that ice forms along evaporator coil 140 blocking some or all of helical pathway 400. Under ideal operating conditions, there will be little to no water in temporary storage space 230. However, in the event that ice forms within helical pathway 400, a corresponding amount of water will be received by temporary storage space 230. Tank 100 preferably comprises an internal tank cap bypass valve 250 that is positioned between the internal tank wall and the water inlet tube 240. The internal tank cap bypass valve 250 is normally closed and prevents water from bypassing the cooling coil flow path 400. However, if the evaporator coil 140 causes ice to build up within helical pathway 400 substantial enough such that the water path in proximity to the evaporator coil 140 is blocked, then bypass valve 250 will allow water to be drawn from the temporary storage space 230 without first traveling along helical path 400. This is designed to prevent the tank 100 from becoming pressurized and being damaged in the event of ice buildup. As shown in FIG. 2, the valve 250 preferably has an umbrella shape and is made of flexible polymeric material allowing the valve 250 to flex under pressure and let water pass by. Water can then go directly from temporary storage space 230 to the water outlet 122. Water will continue to be drawn from the tank 100 in this manner until the ice within helical pathway 400 melts enough to permit water to once again travel along helical pathway 400 before exiting the tank 100.

As shown in FIG. 2, the tank 110 may comprise a sensor well 150. The sensor well 150 preferably extends into the volume of water being maintained by the helical pathway 400 and comprises a sensor capable of determining when the temperature of the water has exceeded a desired temperature. Preferably, the sensor well 150 comprises a capillary tube (a control capillary tube) that maintains a small amount of refrigerant. The sensing portion of the capillary tube is wrapped into a spring like shape in order to get a large amount of the control capillary tube into a small and very focused space of the well 150. At the other end of the control capillary tube is a bellows that is in contact with a mechanical switch. As the temperature at the spring like sensing portion of the control capillary tube becomes warm, the bellows expands, turns on the switch, and in turn sends electricity to the compressor activating the compressor driven refrigeration system in turn cooling the water in the tank 100.

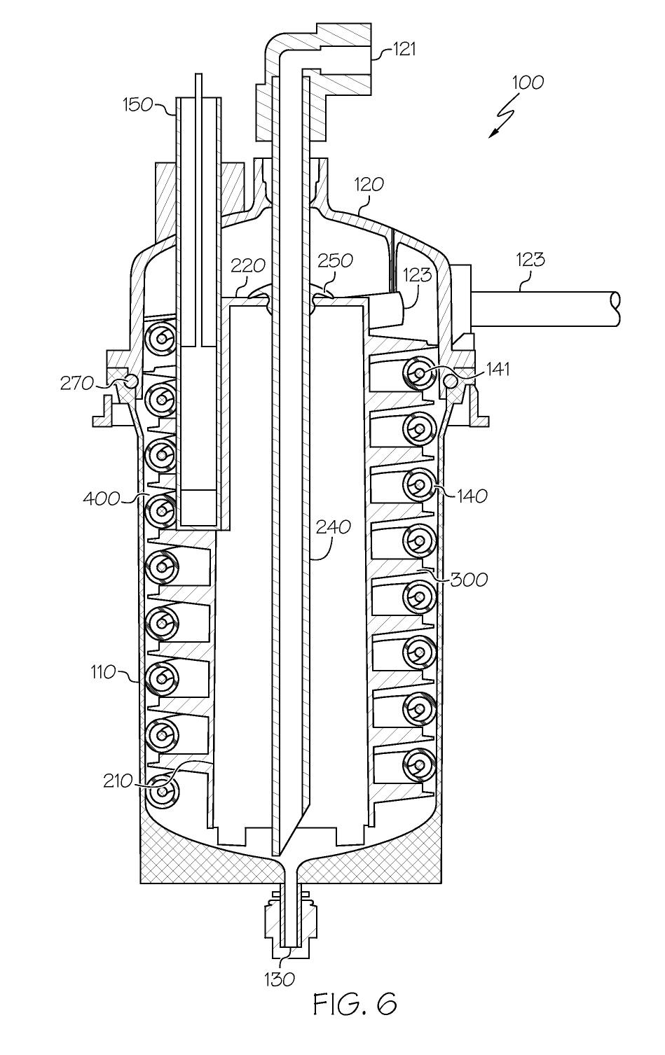

In some exemplary embodiments, such as that shown in FIG. 6, a polymeric material such as high density polyethylene (HDPE) is utilized in the construction of the tank 100. For example, in a preferred exemplary embodiment, the external tank body 110, the internal tank wall 210 (including the helical fin 300), and the exterior cap 120 are all made of HDPE. Such a construction is possible because chilling the water within the tank 100 occurs primarily by placing the water in direct contact with the evaporator coil 140 as the water travels from the water inlet 121 along the helical path 400 and is no longer entirely dependent upon heat transfer through the wall of the water reservoir as was the case with prior art systems. HDPE is less expensive than stainless steel and manufacturing the tank 100 out of HDPE as opposed to stainless steel avoids having to utilize expensive and specialized welding processes. Thus manufacturing the tank 100 is easier and less costly than manufacturing prior art water reservoirs. FIG. 4 shows an exploded front view of the exemplary embodiment that is shown in FIGS. 1, 2, and 3. The arrows in FIG. 4 illustrate the direction that water would preferably flow through the exemplary device when assembled.

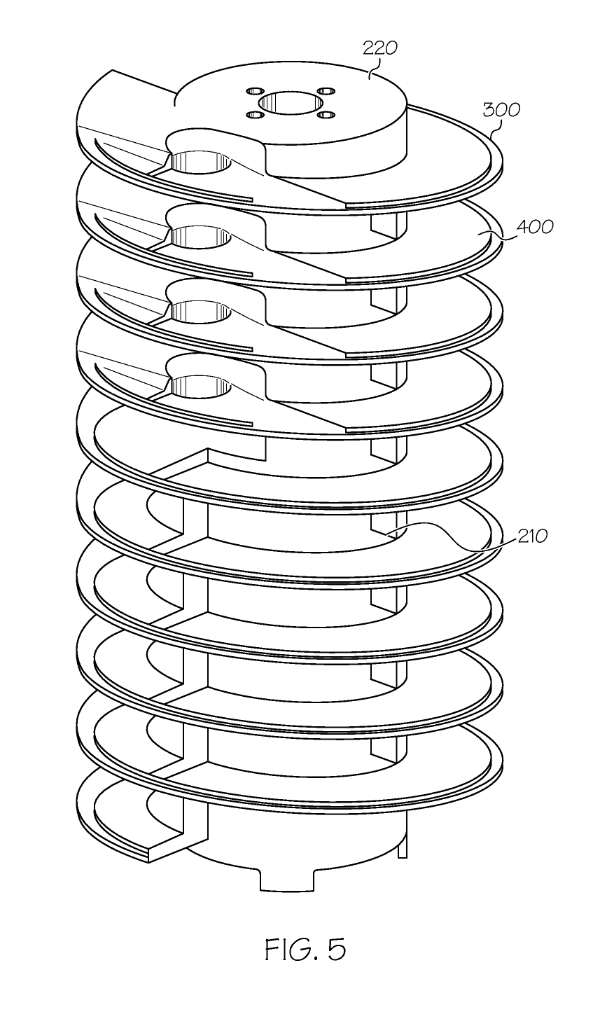

As shown in FIG. 5, in a preferred exemplary embodiment the internal tank wall 210 comprises a helical fin (also referred to as a baffle) 300 that is integral with the internal tank wall 210. As discussed, the internal tank wall 210 and the helical fin 300 are preferably made from plastic such as HDPE. As shown, the internal tank wall 210 and the helical fin 300 may be a single, integrated polymeric unit. FIG. 6 shows a front section view of an exemplary tank 100 utilizing the exemplary internal tank wall 210 that is shown in FIG. 5. As can be seen, in FIG. 6, the helical fin 300 preferably has a width such that the external edge of the helical fin 300 is in contact with the internal surface of the external tank body 110 when the internal tank wall 210 is disposed within the external tank body 110. This creates a helical path 400 defined primarily by the helical fin 300, the interior surface of the external tank body 110, and the exterior surface of the internal tank wall 210. Evaporator coil 140, which is connected to a compressor driven refrigeration system (not shown), is disposed within the helical path 400. In some exemplary embodiments, a helical fin 300 may be a separate piece from the internal tank 210 wall. In other exemplary embodiments, helical fin 300 may be part of the external tank body 110. Other exemplary embodiments may not comprise a helical fin 300 but may rather utilize only an evaporator coil 140 wrapped about the internal tank wall in a helical path wherein the evaporator coil 140 has a width sufficient to span from the internal tank wall 210 to the external tank body 110. In such an exemplary embodiment, the evaporator coil 140 in conjunction with the internal tank wall 210 and external tank body 110 may define a helical pathway 400 for water to travel within the tank 100 being exposed to the evaporator coil 140 so that the water may be chilled. However, embodiments wherein the internal wall 210 comprises a helical fin 300 may be preferable as the helical fin 300 can be made to form a seal with the interior surface of the external tank body 110 better directing flowing water about the helical path 400 and ensuring better exposure of the water to more surface area of the evaporator coil 140.

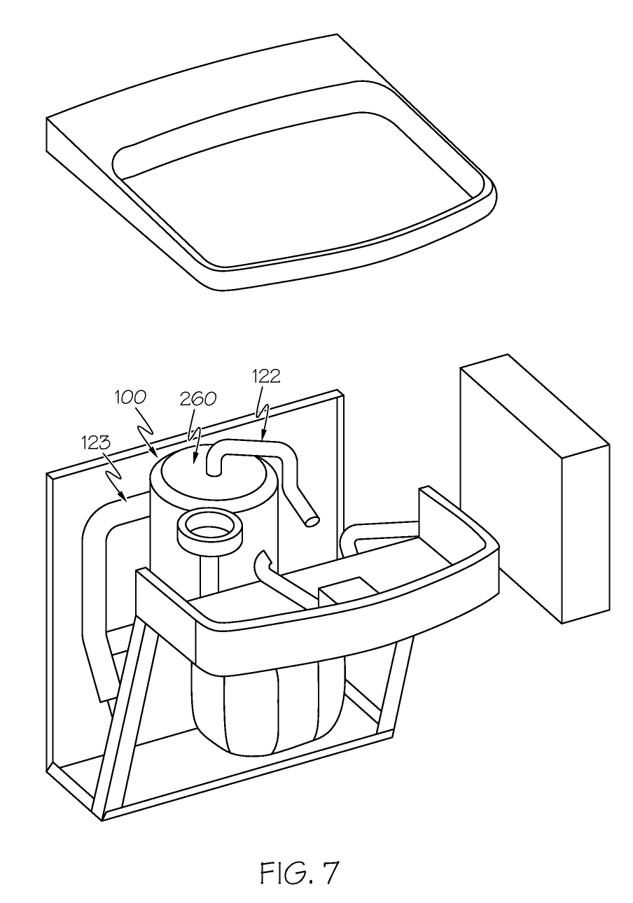

FIG. 7 shows an exploded view of a plumbed water cooler utilizing an exemplary embodiment of a water cooling tank 100. The cooling tank 100 is shown surrounded by insulation 260. FIG. 7 illustrates one example of how the water outlet 122 of the tank 100 may be connected to the dispenser of the water cooler and also provides an example of how the refrigeration inlet/outlet 123 of the tank 100 may be connected to the compressor driven refrigeration unit of the water cooler.

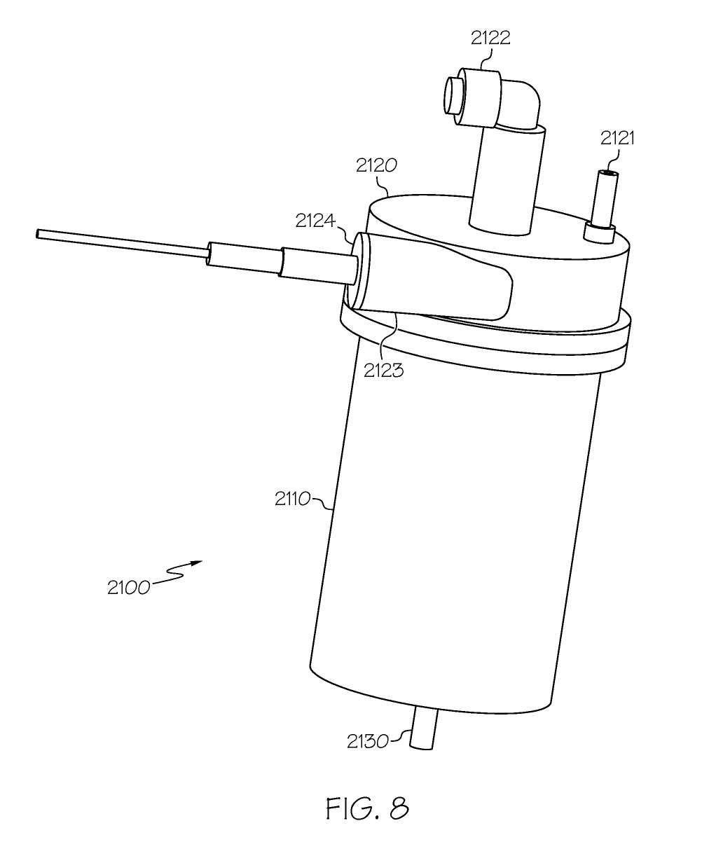

FIG. 8 shows a front perspective view of a second exemplary embodiment of a helical cooling tank 2100. The helical cooling tank 2100 is dual-walled as was the preferred exemplary embodiment shown in FIGS. 1 through 4 and similarly incorporates an evaporator coil within a portion of the tank which receives and at least temporarily maintains a volume of water in order to expose the water to the coil for chilling within the tank. As can be seen, the exemplary tank 2100 of FIG. 8 comprises an external tank body 2110 connected to an external tank cap 2120. External tank cap 2120 comprises/defines a water outlet 2122, a water inlet 2121, and a refrigeration inlet/outlet 2123. Though not shown in the figure, it will be understood that in operation within a water cooler, water inlet 2121 would be connected to and receive water from a water source. Similarly, water outlet 2122 would be connected to a water dispensing means of the water cooler which would permit for chilled water to be removed from the cooling tank 2100. Preferably, the refrigeration inlet/outlet 2123 is connected to a compressor driven refrigeration system which supplies refrigerant to and receives refrigerant from the cooling tank 2100 when water within the cooler is above a desired temperature. The external tank body may comprise a service drain 2130, which permits for water to be drained from the cooling tank 2100 when the tank needs to be serviced, the drain 2130 is preferably located at the bottom of tank 2100. A top cap nut 2124 may be utilized to secure the refrigeration inlet/outlet 2123 within the external cap 2120. It will be appreciated that these features are similar or the same to those of exemplary tank 100 shown in FIGS. 1 through 4 however in exemplary tank 2100 shown in FIG. 8, the water outlet 2122 and water inlet 2121 have been switched from water outlet 122 and water inlet 121 of exemplary tank 100.

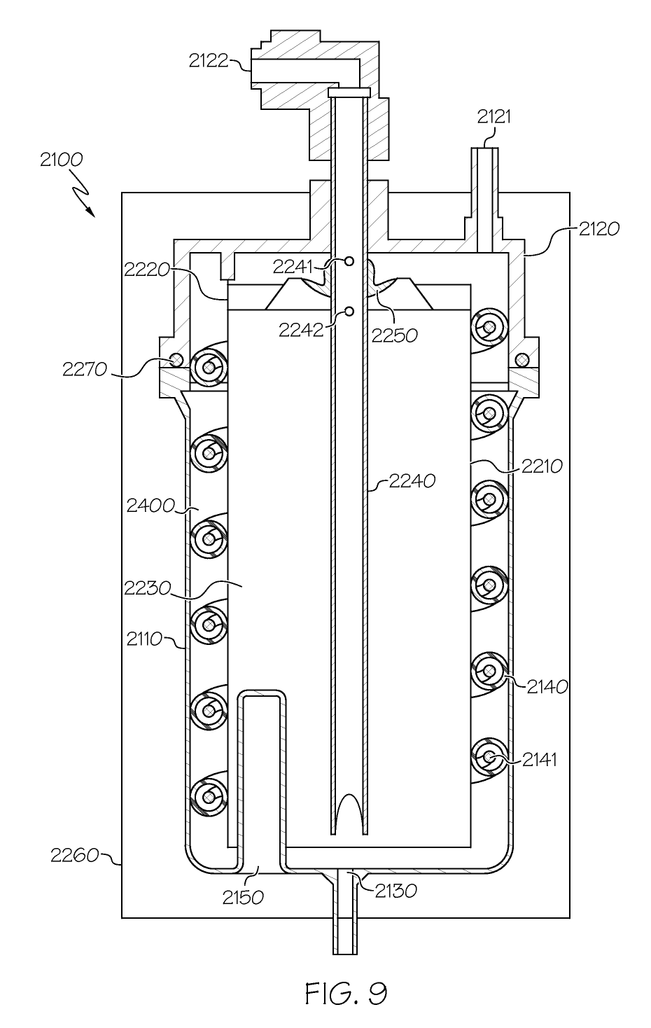

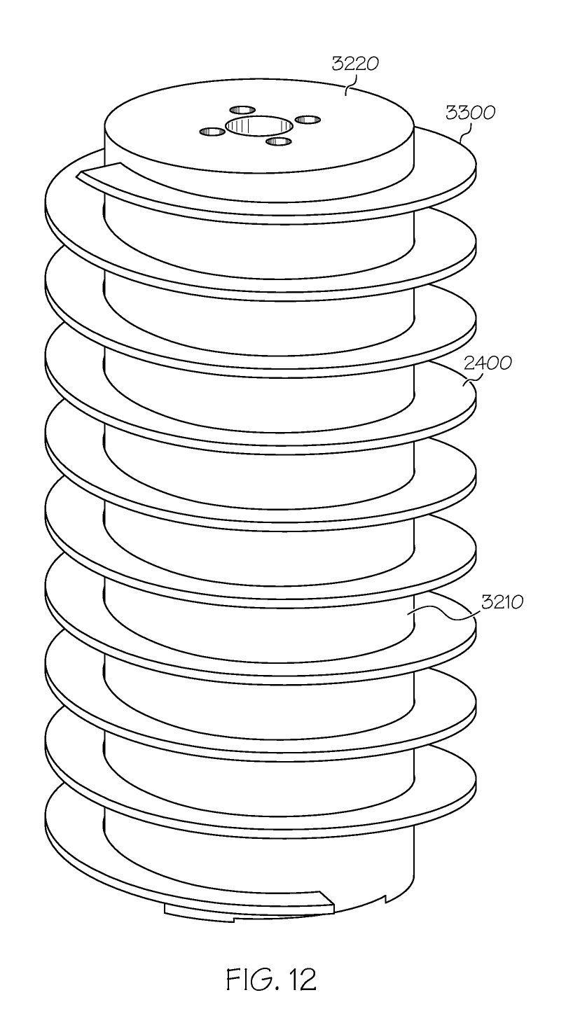

As can be seen in FIG. 9, tank 2100 additionally comprises an internal tank wall 2210 that is disposed within external tank body 2110. External tank cap 2120 preferably comes into contact with both the inner tank wall 2210 and the external tank body 2110 securing the inner tank wall 2210 in a desired position within the external tank body. An o-ring seal 2270 may be positioned between the connection of the external tank cap 2120 and the external tank body 2110 creating a water tight seal. In conjunction with switching the water outlet 2122 and water inlet 2121 of tank 2100, water flows into tank 2100 via inlet 2121 and is received by a space defined at least partially by external cap 2120. As shown in FIG. 9, inner tank wall 2210 may be connected to internal cap 2220 wherein said cap 2220 assists in defining the space for receiving water as it initially enters the tank 2100 via water inlet 2121. In other exemplary embodiments however, and as is actually preferred, inner tank wall 2210 will comprise cap 2220 as a single, integrated inner tank 2210. Such an exemplary embodiment is shown in FIG. 12. In some embodiments, the inner tank wall 2210 may be made of copper or some other metallic material/combination of metallic materials in order to aid in heat transfer from the water to the evaporator coil 2140.

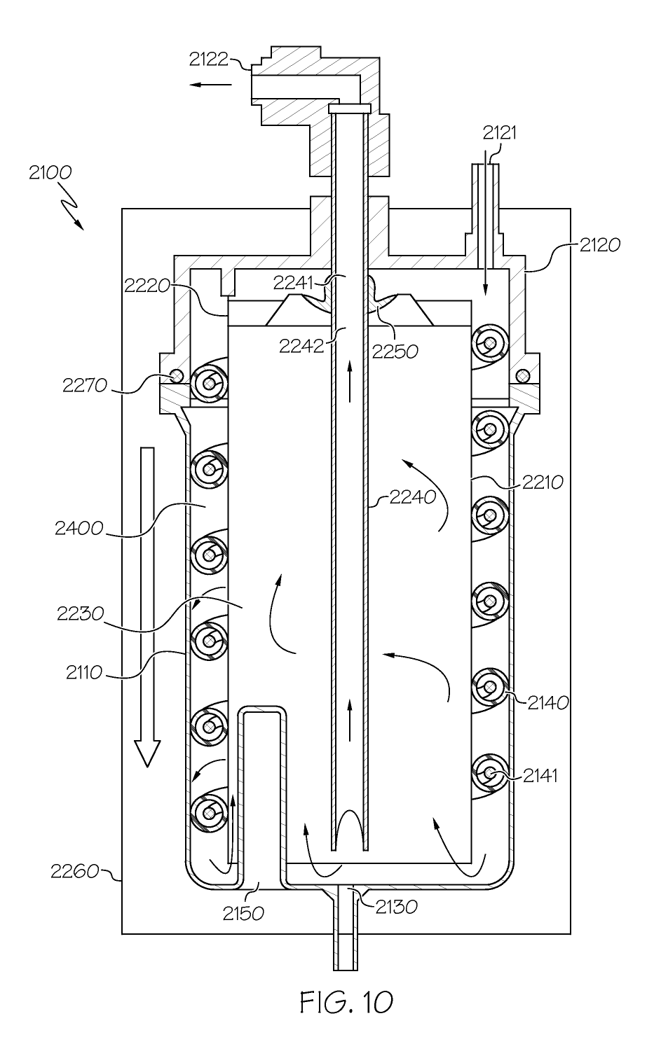

As shown in FIG. 9, the external tank body 2110 and internal tank wall 2210 define a space for receiving at least part of an evaporator coil 2140 that is connected to a compressor driven refrigeration system (or similar cooling system). The refrigeration system is not shown in the Figure. Evaporator coil 2140 is preferably wrapped around internal tank wall 2210 in a helical manner. The evaporator coil 2140 may house a capillary tube 2141 which carries refrigerant into and out of the coil 2140. As shown in the FIG. 9 exemplary embodiment, the evaporator coil 2140 may have a width or diameter that permits for the evaporator coil to span from the internal tank wall 2210 to the interior surface of the external tank body 2110 thereby creating a helical pathway 2400 which may receive water after the water has entered tank 2100 via water inlet 2121. The water may then travel along the helical pathway 2400 where it is exposed to and may be chilled by the evaporator coil 2140. Internal tank wall 2210 and inner tank cap 2220 may define a cold water storage space 2230 which receives and holds a volume of water after the water has traveled the entirety of helical path 2400. FIG. 10, uses arrows to illustrate how water would flow through exemplary tank 2100 under normal operating conditions becoming more chilled as the water travels the length of helical path 2400 due to the water's exposure to the evaporator coil 2140. Exemplary tank 2100 preferably comprises a water outlet tube 2240 which extends from the water outlet 2122 through external cap 2120 and through internal cap 2220 into the cold water storage space 2230. The water outlet tube 2240 permits for chilled water to be drawn from the cold water storage space 2230 to the water outlet where it may then proceed to be dispensed from the water cooler. Insulation 2260 may be utilized around and surrounding the external tank body 2110 to prevent heat transfer between the tank 2100 and the warmer environment within surrounding parts of the water cooler.

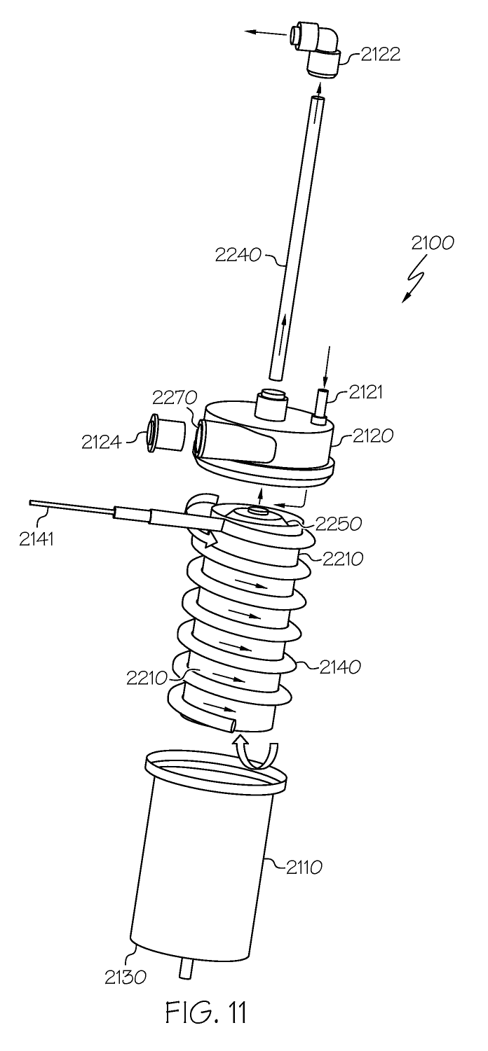

As can be seen in FIG. 9, exemplary tank 2100 may further comprise an inner tank cap by-pass valve 2250. Under normal operating conditions, the valve 2250 will remain closed thereby prohibiting water that has just entered the tank 2100 from entering the cold water storage space 2230 without first traveling along helical pathway 2400. However, if ice buildup occurs along helical pathway 2400 such that water flow along pathway 2400 is inhibited, pressure within the tank may increase enough to cause valve 2250 to open permitting water that has just entered the tank 2100 via water inlet 2121 to access the cold water storage space 2230 without first traveling along helical pathway 2400. As was the case with valve 250 of exemplary tank 100 which was previously discussed, valve 2250 is preferably made from a polymeric material flexible enough to morph under a certain amount of pressure that will be reached inside the tank when ice build-up blocks helical pathway 2400. Valve 2250 is also preferably umbrella-like in shape. However, valve 2250 is positioned in the opposite direction compared to valve 250 in light of and to account for the fact that the water inlet 2121 and water outlet 2122 are reversed from that of exemplary tank 100. FIG. 11 shows an exploded view of the exemplary tank 2100 shown in FIGS. 8, 9, and 10. FIG. 11 uses arrows to illustrate how water would flow through exemplary tank 2100 under normal operating conditions.

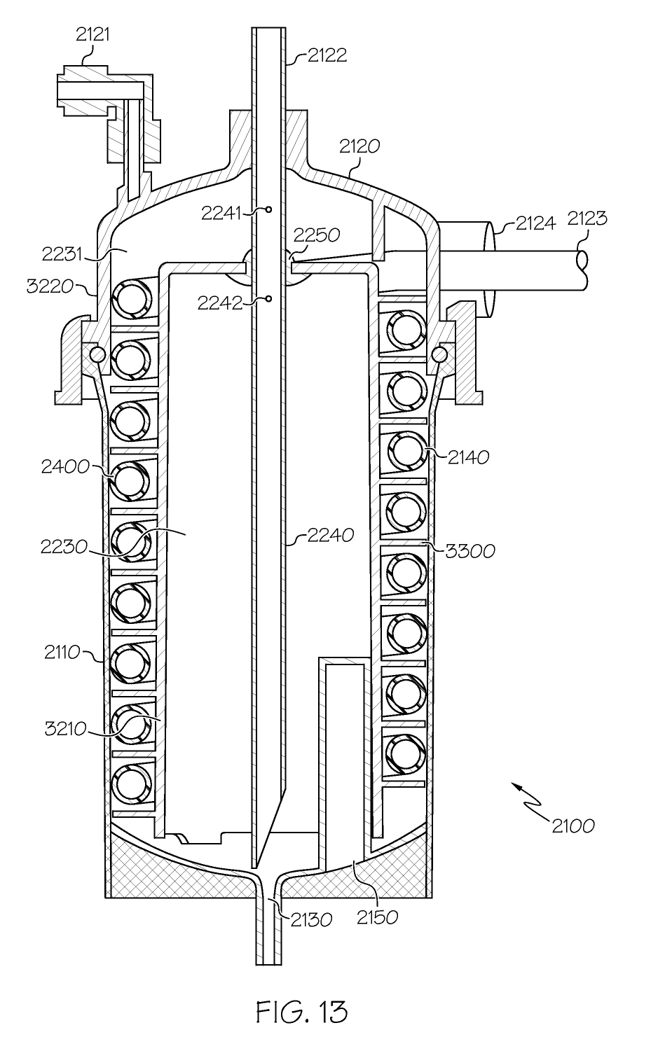

In some exemplary embodiments, exemplary tank 2100 may comprise an exemplary inner tank wall 3210 as is shown in FIG. 12. The exemplary inner tank wall 3210 shown in FIG. 12 is a single, integrated piece comprising a helical fin 3300 and inner tank cap 3220. FIG. 13 shows an exemplary embodiment of tank 2100 utilized in conjunction with exemplary internal wall 3210. When tank 2100 comprises inner tank wall 3210, the helical fin 3300 preferably extends to the interior surface of the external tank body 2110 as is shown in FIG. 13 thereby defining helical pathway 2400. Evaporator coil 2140 is preferably disposed along helical pathway 2400 but because helical fin 3300 extends to the interior surface of the external tank body 2110, it is not necessary for the evaporator coil 2140 to have intimate contact with both the internal tank wall 3210 and the interior surface of the external tank body 2110. This is because helical pathway 2400 is defined by the helical fin 3300 in conjunction with the internal tank wall 3210 and the interior surface of the external tank body 2110 providing water that has entered the tank 2100 via the water inlet 2121 with a pathway to travel and be exposed to the evaporator coil 2140. Such an embodiment may be preferred because it may permit for a greater surface area of the evaporator coil 2140 to be in contact with water within helical pathway 2400 leading to more effective chilling of the water.

As is visible in FIG. 13, the water outlet tube 2240 of exemplary tank 2100 may define a first air vent hole 2241 and a second air vent hole 2242 that allow air to escape from the tank 2100 when a volume of water is introduced to the tank 2100 via the water inlet 2121. The first air vent hole 2241, which is located along the portion of the water outlet tube 2240 that extends between the external cap 2120 and the internal cap 3220, permits for air to escape from space 2231 as the tank 2100 receives water from the water inlet 2121. The second air vent hole 2242, which is located along the portion of the water outlet tube 2240 that extends through bypass valve 2250 into space 2230 between the internal cap 3220 and the internal tank cap bypass valve 2250, permits for air to escape from the cold water storage space 2230 as it receives water that has traveled the helical path along the evaporator coil 2140. After entering the water outlet tube 2240 via air vent holes 2241 and/or 2242, air may escape the tank 2100 via passage through the water outlet tube 2240 and water outlet 2122.

Exemplary tank 2100 may comprise a sensor well 2150. As can be seen in FIG. 9, the sensor well 2150 is preferably disposed within cold water storage space 2230 permitting the sensor well 2150 to gauge the temperature of the water being housed within the space 2230. When the sensor within the well 2150 detects that the temperature of the water within the storage space 2230 has risen above a desired temperature, the sensor preferably communicates with the compressor driven refrigeration system causing it to turn on and supply refrigerant to the evaporator coil 2140. This in turn causes the temperature of the water within the tank 2100 to drop.

Any embodiment of the disclosed system and method may include any of the optional or preferred features of the other embodiments of the present invention. The exemplary embodiments herein disclosed are not intended to be exhaustive or to unnecessarily limit the scope of the invention. The exemplary embodiments were chosen and described in order to explain the principles of the present invention so that others skilled in the art may practice the invention. Having shown and described exemplary embodiments of the present invention, those skilled in the art will realize that many variations and modifications may be made to affect the described invention. Many of those variations and modifications will provide the same result and fall within the spirit of the claimed invention. It is the intention, therefore, to limit the invention only as indicated by the scope of the claims.

1. A method for selecting a full length antibody comprising the following steps:
a) generating from a parent full length antibody a plurality of full length antibodies by randomizing one or more amino acid residues selected from the amino acid residues at positions 1-23 in the heavy chain variable domain (numbering according to Kabat), at positions 55-83 in the light chain variable domain (numbering according to Kabat), at positions 145-174 in the first heavy chain constant domain (numbering according to EU index) and at positions 180-197 in the first heavy chain constant domain (numbering according to EU index),
b) determining the binding strength of each of the full length antibodies from the plurality of antibodies to the human neonatal Fc receptor (FcRn), and
c) selecting a full length antibody from the plurality of full length antibodies that has a different binding strength to the FcRn than the parent full length antibody.

2. A plurality of full length antibodies generated from a single full length antibody by randomizing one or more amino acid residues selected from the amino acid residues at positions 1-23 in the heavy chain variable domain (numbering according to Kabat), at positions 55-83 in the light chain variable domain (numbering according to Kabat), at positions 145-174 in the first heavy chain constant domain (numbering according to EU index) and at positions 180-197 in the first heavy chain constant domain (numbering according to EU index).

3. Use of one or more amino acid mutations at positions selected from the group of positions comprising positions 1-23 in the heavy chain variable domain (numbering according to Kabat), positions 55-83 in the light chain variable domain (numbering according to Kabat), positions 145-174 in the first heavy chain constant domain (numbering according to EU index) and positions 180-197 in the first heavy chain constant domain (numbering according to EU index) for changing the in vivo half-life of a full length antibody.

4. A variant full length antibody comprising two light chain polypeptides and two heavy chain polypeptides, wherein the variant antibody is derived from a parent full length antibody by introducing amino acid mutations at one or more positions selected from the group of positions comprising positions 1-23 in the heavy chain variable domain (numbering according to Kabat), positions 55-83 in the light chain variable domain (numbering according to Kabat), positions 145-174 in the first heavy chain constant domain (numbering according to EU index) and positions 180-197 in the first heavy chain constant domain (numbering according to EU index), and wherein the variant antibody has a different affinity for the human neonatal Fc receptor than the parent full length antibody.

5. The antibody according to claim 4, wherein the one or more amino acid residues are selected from the amino acid residues at positions 5-18 in the heavy chain variable domain (numbering according to Kabat).

6. The antibody according to claim 4, wherein the one or more amino acid residues are selected from the amino acid residues at positions 145-174 in the first heavy chain constant domain (numbering according to EU index).

7. The antibody according to claim 4, wherein the one or more amino acid residues are selected from the amino acid residues at positions 161-174 in the first heavy chain constant domain (numbering according to EU index).

8. The antibody according to claim 4, wherein the one or more amino acid residues are selected from the amino acid residues at positions 181-196 in the first heavy chain constant domain (numbering according to EU index).

9. The antibody according to claim 4, wherein the one or more amino acid residues are selected from the amino acid residues at positions 182-197 in the first heavy chain constant domain (numbering according to EU index).

10. The antibody according to claim 4, wherein the one or more amino acid residues are selected from the amino acid residues at positions 55-83 in the light chain variable domain (numbering according to Kabat).

11. The antibody according to claim 4, wherein the one or more amino acid residues are selected from the amino acid residues at positions 55-73 in the light chain variable domain (numbering according to Kabat).

12. The antibody according to claim 4, wherein the one or more amino acid residues are selected from the amino acid residues at positions 57-70 in the light chain variable domain (numbering according to Kabat).

13. The antibody according to claim 4, wherein the antibody is a full length IgG antibody.

14. The antibody according to claim 13, wherein the antibody is a full length IgGI antibody or a full length IgG4 antibody.

15. The antibody according to claim 4, wherein the mutation is a mutation from the amino acid residue to a different amino acid residue from the same group of amino acid residues.

16. The antibody according to claim 4, wherein one or more of the following mutations are introduced (numbering according to Kabat variable domain numbering and Kabat EU index numbering scheme, respectively):
heavy chain E6Q,
heavy chain A162D,
heavy chain A162E,
heavy chain T164D,
heavy chain T164E,
heavy chain S165D,
heavy chain S165E,
heavy chain S191D,
heavy chain S191E,
heavy chain G194D,
heavy chain G194E,
heavy chain T195D,
heavy chain T195E,
heavy chain Q196D,
heavy chain Q196E,
light chain G57K,
light chain G57R,
light chain S60K, and
light chain S60R.


SEQUENCE LISTING (14 sequences)

SEQ ID NO: 1 - protein, 125 aa, Artificial Sequence
  humanized anti-digoxigenin antibody VH
QVQLVESGGGLVKPGGSLRLSCAASGFTFSDYAMSWIRQAPGKGLEWVSSINIGATYIYYADSVKGRFTISRDNAKNSLYLQMNSLRAEDTAVYYCARPGSPYEYDKAYYSMAYWGQGTTVTVSS

SEQ ID NO: 2 - protein, 330 aa, Homo sapiens [sequence omitted]

SEQ ID NO: 3 - protein, 107 aa, Artificial Sequence
  humanized anti-digoxigenin antibody VL
DIQMTQSPSSLSASVGDRVTITCRASQDIKNYLNWYQQKPGKAPKLLIYYSSTLLSGVPSRFSGSGSGTDFTLTISSLQPEDFATYYCQQSITLPPTFGGGTKVEIK

SEQ ID NO: 4 - protein, 107 aa, Homo sapiens [sequence omitted]

SEQ ID NO: 5 - protein, 115 aa, Artificial Sequence [sequence omitted]

SEQ ID NO: 6 - protein, 112 aa, Artificial Sequence
  Briakinumab VL amino acid sequence
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLG

SEQ ID NO: 7 - protein, 119 aa, Artificial Sequence
  Ustekinumab VH amino acid sequence
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSS

SEQ ID NO: 8 - protein, 107 aa, Artificial Sequence
  Ustekinumab VL amino acid sequence
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIK

SEQ ID NO: 9 - protein, 274 aa, Homo sapiens
AESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSS

SEQ ID NO: 10 - protein, 99 aa, Homo sapiens [sequence omitted]

SEQ ID NO: 11 - protein, 38 aa, Homo sapiens
GGTAALGCLVKDYFPEPVTVSWNSGALTSGVHTFPAVL

SEQ ID NO: 12 - protein, 14 aa, Homo sapiens
GALTSGVHTFPAVL

SEQ ID NO: 13 - protein, 18 aa, Homo sapiens
YSLSSVVTVPSSSLGTQT

SEQ ID NO: 14 - protein, 16 aa, Homo sapiens
LSSVVTVPSSSLGTQT